(19) United States
(12) Patent Application Publication
(10) Pub. No.: US 2025/0069372 A1
(43) Pub. Date: Feb. 27, 2025

(54) STRESS ESTIMATION DEVICE, STRESS ESTIMATION METHOD, AND STORAGE MEDIUM

(71) Applicant: NEC Corporation, Minato-ku, Tokyo (JP)

(72) Inventor: Masanori Tsujikawa, Tokyo (JP)

(73) Assignee: NEC Corporation, Minato-ku, Tokyo (JP)

(21) Appl. No.: 18/725,900

(22) PCT Filed: Jan. 11, 2022
(86) PCT No.: PCT/JP2022/000515

§ 371 Date: Jul. 1, 2024

(51) Int. Cl. G06V 10/77; G06V 10/98; G06V 40/20

(52) U.S. Cl. CPC G06V 10/7715; G06V 10/993; G06V 40/20

(57) ABSTRACT

The stress estimating device 1X mainly includes a division means 15X, a first feature value calculation means 16X, a second feature value calculation means 17X, and a stress estimation means 18X. The division means 15X is configured to divide observation data representing chronological states of a subject. The first feature value calculation means 16X is configured to calculate first feature values, which are feature values of divided observation data, based on a learned feature extraction model. The second feature value calculation means 17X is configured to calculate a second feature value based on a plurality of the first feature values. The stress estimation means 18X is configured to estimate a stress of the subject based on the second feature value.

### TECHNICAL FIELD

The present disclosure relates to a technical field of a stress estimation device, a stress estimation method, and a storage medium configured to perform processing related to estimation of a stress state.

### BACKGROUND

There is a device or a system for determining a stress state of a subject based on data measured from the subject. For example, Patent Literature 1 discloses a stress estimation device for estimating a stress value (stress level) of a subject based on biological data of the subject.

### CITATION LIST

### Patent Literature

Patent Literature 1: WO2019/159252

### SUMMARY

### Problem to be Solved

In Patent Literature 1, it is assumed that features to be extracted for estimating a stress value of a subject from biological data of the subject are manually designed. On the other hand, there is an issue that sufficiently-characteristic information could not be extracted from biological data in using such features designed manually.

In view of the above-described issues, it is an object of the present disclosure to provide a stress estimation device, a stress estimation method, and a storage medium capable of suitably estimating a stress of a subject.

### Means for Solving the Problem

In one aspect of the stress estimation device, there is provided a stress estimation device including:
a division means configured to divide observation data representing chronological states of a subject;
a first feature value calculation means configured to calculate first feature values, which are feature values of the divided observation data, based on a learned feature extraction model;
a second feature value calculation means configured to calculate a second feature value based on a plurality of the first feature values; and
a stress estimation means configured to estimate a stress of the subject based on the second feature value.

In one aspect of the stress estimation method, there is provided a stress estimation method including:
dividing observation data representing chronological states of a subject;
calculating first feature values, which are feature values of the divided observation data, based on a learned feature extraction model;
calculating a second feature value based on a plurality of the first feature values; and
estimating a stress of the subject based on the second feature value.

In one aspect of the storage medium, there is provided a storage medium storing a program executed by a computer, the program causing the computer to:
divide observation data representing chronological states of a subject;
calculate first feature values, which are feature values of the divided observation data, based on a learned feature extraction model;
calculate a second feature value based on a plurality of the first feature values; and estimate a stress of the subject based on the second feature value.

### Effect

An example advantage according to the present invention is to estimate stress of the subject with high accuracy.

### BRIEF DESCRIPTION OF THE DRAWINGS

FIG. 1 It illustrates a schematic configuration of a stress estimation system according to a first example embodiment.

FIG. 2 It illustrates an example of a hardware configuration of a stress estimation device common to each example embodiment.

FIG. 3 It is an example of a functional block diagram of the stress estimation device according to the first example embodiment.

FIG. 4 It is an example of a detailed functional block diagram of a short-term feature value calculation unit.

FIG. 5 It schematically illustrates a specific example of the process executed by the short-term feature value calculation unit.

FIG. 6 It is an example of a flowchart showing a procedure of the stress estimation process performed by the stress estimation device.

FIG. 7 It is a functional block diagram of a short-term feature value calculation unit according to a first modification.

FIG. 8 It is a functional block diagram of a short-term feature value calculation unit according to a second modification.

FIG. 9 It is a functional block diagram relating to the learning of a dimension reduction model.

FIG. 10 It illustrates a schematic configuration of a stress estimation system in a second example embodiment.

FIG. 11 It is a block diagram of a stress estimation device according to a third example embodiment.

FIG. 12 It is an example of a flowchart executed by the stress estimation device in the third example embodiment.

### EXAMPLE EMBODIMENTS

Hereinafter, example embodiments of a stress estimation device, a stress estimation method, and a storage medium will be described with reference to the drawings.

### First Example Embodiment

### (1) System Configuration

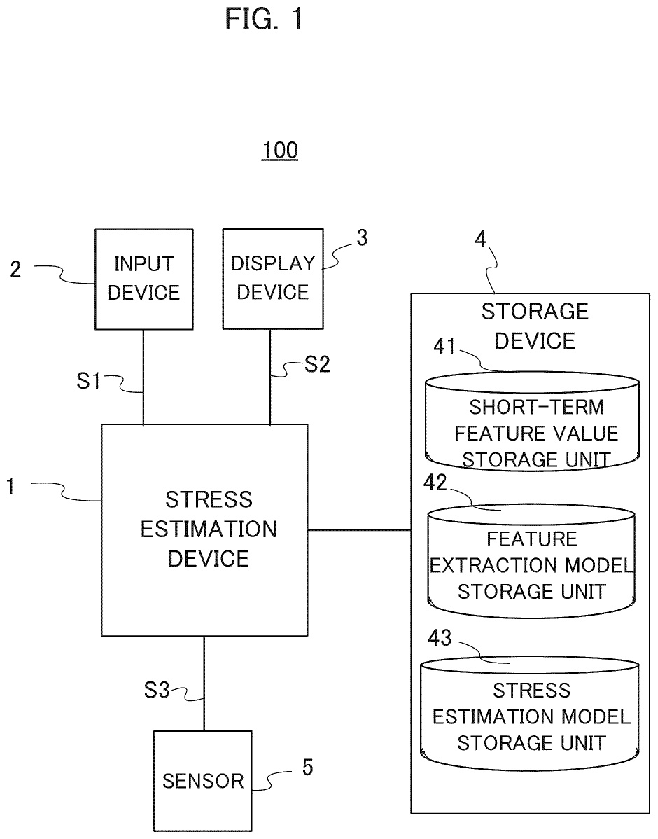

FIG. 1 shows a schematic configuration of a stress estimation system 100 according to a first example embodiment. The stress estimation system 100 is a system for estimating stress of a subject based on data observed from the subject, and mainly includes a stress estimation device 1, an input device 2, a display device 3, a storage device 4, and a sensor 5. The term “subject” herein indicates a subject of stress estimation, and may be a sports player or an employee subjected to management of the stress state by the organization, or may be an individual user. Further, the stress estimated in the present example embodiment is assumed to be a chronic stress which is a stress in a long-term (chronic) perspective on a several-days, weekly or monthly basis.

The stress estimation device 1 estimates the stress state (specifically, a stress value representing the degree of stress) of the subject. In this case, the stress estimation device 1 exchanges data relating to stress estimation process with the input device 2, the display device 3, and the sensor 5 through a communication network or through wireless or wired direct communication. For example, the stress estimation device 1 receives the input signal “S1” from the input device 2. In addition, the stress estimation device 1 receives observation data “S3” representing an observation result by the sensor 5 from the sensor 5 which senses the subject. In addition, the stress estimation device 1 generates a display signal “S2” based on the estimation result of the stress value of the subject and supplies the generated display signal S2 to the display device 3.

The input device 2 is one or more interfaces that receive user input (manual input) of information regarding each subject. The user who inputs information using the input device 2 may be the subject itself, or may be a person who manages or supervises the activity of the subject. The input device 2 may be a variety of user input interfaces such as, for example, a touch panel, a button, a keyboard, a mouse, and a voice input device. The input device 2 supplies the input signal S1 generated based on the input from the user to the stress estimation device 1. The display device 3 displays information based on the display signal S2 supplied from the stress estimation device 1. Examples of the display device 3 include a display and a projector.

The sensor 5 generates observation data S3 that is data obtained by observing the subject and supplies the generated observation data S3 to the stress estimation device 1. For example, the observation data S3 is any biological signal (including vital information) such as heartbeat, EEG, amount of perspiration, amount of hormonal secretion, cerebral blood flow, blood pressure, body temperature, electromyogram, electrocardiogram, respiration rate, pulse wave, acceleration regarding the subject. The sensor 5 may be a wearable terminal worn by the subject, or may be a camera for photographing the subject or a microphone for generating a voice signal of the utterance of the subject, or may be any sensor mounted on a terminal such as a personal computer and a smartphone operated by the subject. For example, the above-described wearable terminal includes a GNSS (global navigation satellite system) receiver, an accelerometer, and/or any other sensors that detect biological signals, and outputs output signals from the respective sensors as observation data S3. The sensor 5 may supply information corresponding to the operation quantity of the personal computer or the smart phone to the stress estimation device 1 as the sensor signal S3. The sensor 5 may output observation data S3 representing biological data (including sleep time) of the subject during sleep of the subject. Time information (time stamp) representing the observation time is associated with the observation data S3 by the sensor 5 or the stress estimation device 1 that has received the observation data S3.

The storage device 4 is one or more memories for storing various information necessary for estimating the stress state. The storage device 4 may be an external storage device, such as a hard disk, connected or embedded in the stress estimation device 1, or may be a storage medium, such as a flash memory. The storage device 4 may be a server device that performs data communication with the stress estimation device 1. Further, the storage device 4 may be configured by a plurality of devices.

The storage device 4 functionally includes a short-term feature value storage unit 41, a feature extraction model storage unit 42, and a stress estimation model storage unit 43.

The short-term feature value storage unit 41 stores short-term feature values which are feature values of data (also referred to as “unit observation data”) per unit time into which the observation data S3 is divided. Here, the unit time described above is set to an arbitrary time length shorter than the observation period (also referred to as “required observation period”) of the subject which is necessary for estimating the stress value (i.e., the degree of chronic stress) of the subject. The required observation period, in other words, is the duration which affects the stress to be estimated. For example, if observation data S3 for one month is used to estimate the subject's stress (i.e., the required observation period is one month period immediately before the stress estimation time), the above-described unit time is set to several minutes (e.g., one minute). The stress estimation device 1 calculates the short-term feature value for each of the unit observation data obtained by dividing the observation data S3 received from the sensor 5, and stores the calculated short-term feature values in the short-term feature value storage unit 41. In this case, as will be described later, the stress estimation device 1 converts the unit observation data into data in a tensor format (image in the present example embodiment) that can be inputted to the feature extraction model whose learned parameters are already stored in the feature extraction model storage unit 42. Then, the stress estimation device 1 calculates the feature values, which are extracted by the feature extraction model from the data after conversion, as the short-term feature values. The short-term feature values are an example of the “first feature values”.

The feature extraction model storage unit 42 stores parameters (in other words, parameters necessary for configuring the feature extraction model) of the feature extraction model that is a model for calculation of the short-term feature values. In the present example embodiment, the feature extraction model is a model that is trained to output a feature value (feature vector) representing features of the image when an image with a predetermined size is inputted to the model. The feature extraction model may be equipped with an architecture of any feature extraction model for general image recognition. For example, as such a feature extraction model, there are various deep-learning models such as VGG16, VGG19, Mobile-Net. Various forms have been proposed as a feature extraction model (feature extractor) using an image as an input, and any form thereof may be used. Then, the feature extraction model storage unit 42 stores the learned parameters of the feature extraction model. For example, when the feature extraction model is a model based on the neural network, the feature extraction model storage unit 42 stores various parameter information regarding the layer structure, the neuron structure of each layer, the number of filters and the filter size in each layer, and the weight for each element of each filter.

The feature extraction model used in this example embodiment may be trained to extract a feature value suitable for this example embodiment. In this case, for example, the feature extraction model is preliminarily trained by using images, into which unit observation data prepared as training data is converted, as input data, and parameters of the feature extraction model obtained by the above training are stored in the feature extraction model storage unit 42 in advance (i.e., before stress estimation of the subject).

The stress estimation model storage unit 43 stores parameters of the stress estimation model which is a model configured to estimate the stress value of the subject (in other words, parameters necessary for configuring the stress estimation model). Here, the feature value to be inputted to the stress estimation model is a feature value representing the statistic of the short-term feature values generated from the unit observation data indicating observation results in the required observation period, and hereinafter also referred to as “stress feature value”. If each of the short-term feature value and the stress feature value is in a vector format, a vector representing statistics (e.g., averaged values) for respective vector elements of the short-term feature values are calculated as the stress feature value. If the required observation period is one month period right before the stress estimation time, the stress feature value is calculated using the short-term feature values extracted from the unit observation data which indicates the observation results during the one-month period. The stress feature value is an example of the “second feature value”.

The stress estimation model is a model which estimates the relation between the stress feature value of the subject and the stress value of the subject, and is trained, in advance, to output the estimated stress value of the subject when the stress feature value of the subject is inputted to the model. Here, the stress estimation model may be any machine learning model (including a statistical model) such as a neural network, a support vector machine, and the like. For example, when the stress estimation model is a model based on a neural network such as a convolutional neural network, the stress estimation model storage unit 43 stores various parameter information regarding the layer structure, the neuron structure of each layer, the number of filters and the filter size in each layer, and the weight for each element of each filter.

The stress estimation model may be a model that is trained for each of predetermined classes of possible subjects. In this case, the parameters of the model for each class are stored in the stress estimation model storage unit 43. In such cases, the above-described classification may be performed based on the subject's attribute and/or the type of the observation data S3 used for stress estimation. The classification based on subject's attribute herein indicates a classification based on personality, gender, job type, race, age, height, weight, muscle weight, stress tolerance, life habit, exercise habit, tendency of cognition, or a combination thereof. Then, in the stress estimation, the stress estimation device 1 determines a stress estimation model to be used based on the attribute information of the subject or/and the type information of the observation data S3. In this way, a stress estimation model may be trained and used for each group that is biased in stress trends.

The configuration of the stress estimation system 100 shown in FIG. 1 is an example, and various changes may be made to the configuration. For example, the input device 2 and the display device 3 may be configured integrally. In this case, the input device 2 and the display device 3 may be configured as a tablet-type terminal that incorporates or is separate from the stress estimation device 1. In this case, the stress estimation device 1, the input device 2, the display device 3, and the sensor 5 (and may include the storage device 4) may be configured as one smartphone or a wearable terminal used by the subject. Further, the stress estimation device 1 may be configured by a plurality of devices. In this case, the plurality of devices constituting the stress estimation device 1 performs transmission and reception of information necessary for executing the process allocated in advance among the plurality of devices.

### (2) Hardware Configuration of Stress Estimation Device

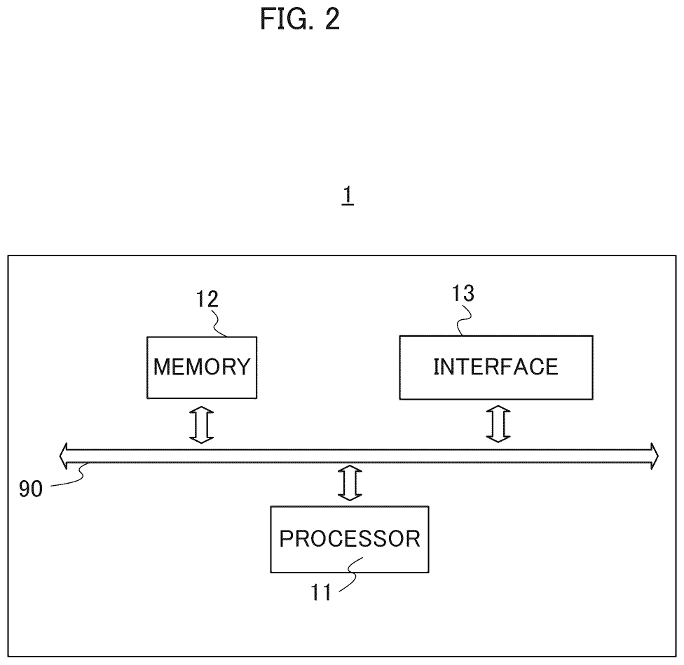

FIG. 2 shows a hardware configuration of the stress estimation device 1. The stress estimator 1 includes a processor 11, a memory 12, and an interface 13 as hardware. The processor 11, memory 12 and interface 13 are connected via a data bus 90 to one another.

The processor 11 functions as a controller (arithmetic unit) that performs overall control of the stress estimation device 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 includes a variety of volatile and non-volatile memories, such as a RAM (Random Access Memory), a ROM (Read Only Memory), and a flash memory. Further, a program for executing a process performed by the stress estimation device 1 is stored in the memory 12. A part of the information stored in the memory 12 may be stored in one or more external storage devices capable of communicating with the stress estimation device 1, or may be stored in a storage medium detachable from the stress estimation device 1.

The interface 13 is one or more interfaces for electrically connecting the stress estimation device 1 to other devices. Examples of these interfaces include a wireless interface, such as a network adapter, for transmitting and receiving data to and from other devices wirelessly, and a hardware interface, such as a cable, for connecting to other devices.

The hardware configuration of the stress estimation device 1 is not limited to the configuration shown in FIG. 2. For example, the stress estimation device 1 may include at least one of the input device 2 and/or the display device 3. In addition, the stress estimation device 1 may incorporate or be connected to a sound output device such as a speaker.

### (3) Overview of Stress Estimation

In summary, the stress estimation device 1 calculates the short-term feature value through image conversion of the unit observation data obtained by dividing the observation data S3 and then estimates the stress value of the subject based on the stress feature value calculated through statistical processing of plural short-term feature values. In this case, the stress estimation device 1 acquires short-term feature values useful for stress estimation by calculating short-term feature values using a learned feature extraction model with an architecture developed for the purpose of image recognition or the like, and thereby realizes high-precision stress estimation.

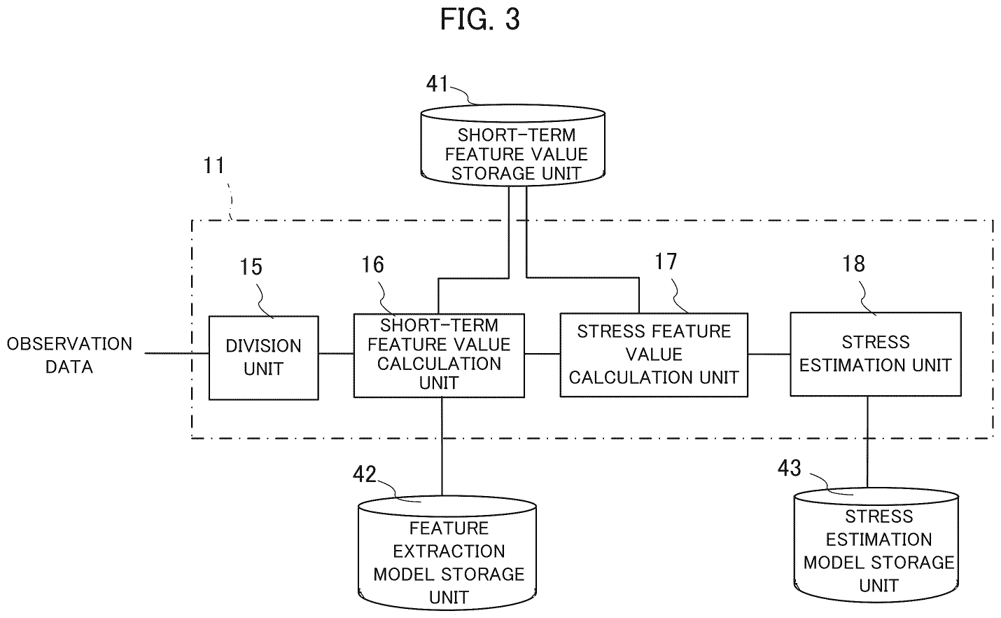

FIG. 3 is an example of a functional block diagram of the stress estimation device 1. The processor 11 of the stress estimation device 1 functionally includes a division unit 15, a short-term feature value calculation unit 16, a stress feature value calculation unit 17, and a stress estimation unit 18. In FIG. 3, blocks to exchange data with each other are connected to each other by a solid line, but the combination of blocks to exchange data with each other is not limited to the combination shown in the drawings. The same applies to the drawings of other functional blocks described below.

The division unit 15 acquires the observation data S3 of the subject supplied from the sensor 5 through the interface 13 and generates unit observation data into which the observation data S3 is divided for each unit time. In other words, the division unit 15 cuts out the unit observation data from the observation data S3 by applying a short-time window with an above-mentioned unit time length to the observation data S3. The observation data S3 generated by the sensor 5 may be acquired and divided by the division unit 15 after temporarily stored in the storage device 4 or the memory 12. The division unit 15 supplies the generated unit observation data to the short-term feature value calculation unit 16.

The short-term feature value calculation unit 16 converts each piece of unit observation data supplied from the division unit 15 into a short-term feature value. In this case, the short-term feature value calculation unit 16 converts the unit observation data so as to conform to the input format of the feature extraction model. Thereafter, the short-term feature value calculation unit 16 configures the learned feature extraction model by referring to the feature extraction model storage unit 42, and inputs data (an image in the present example embodiment), into which the unit observation data is converted, to the feature extraction model. Then, the short-term feature value calculation unit 16 stores each short-term feature value outputted by the feature extraction model in the short-term feature value storage unit 41 in association with the time information included in the corresponding unit observation data. In addition, the short-term feature value calculation unit 16 identifies data with deficiency (that is, does not correctly represent the state of the subject) among the data after the conversion to conform to the input format of the feature extraction model and excludes the data from the target data of input to the feature extraction model. Details of the process executed by the short-term feature value calculation unit 16 will be described later.

In the stress estimation of the subject, the stress feature value calculation unit 17 acquires the short-term feature values corresponding to the required observation period from the short-term feature value storage unit 41 and calculate the stress feature value based on the acquired short-term feature values. The stress feature value is in a vector format, and each vector element of the stress feature value becomes a statistic such as an average of corresponding elements of the acquired short-term feature values. In this case, in some embodiments, the stress feature value calculation unit 17 may calculate a feature value (also referred to as “daily-basis feature value”) representing the statistic of the short-term feature values for each observation date, and then calculate the stress feature values representing the statistic of the daily-basis feature values corresponding to respective dates belonging to the required observation period. Thus, the stress feature value calculation unit 17 may calculate the stress feature values by performing multi-step statistical processing on the target short-term feature values. The stress feature value calculation unit 17 supplies the calculated stress feature value to the stress estimation unit 18.

The stress estimation unit 18 calculates an estimate value (also referred to as “stress estimate value”) of the stress of the subject based on the stress feature value calculated by the stress feature value calculation unit 17. In this case, the stress estimation unit 18 inputs the stress feature value to the stress estimation model configured by referring to the stress estimation model storage unit 43, and acquires the stress value outputted by the stress estimation model as the stress estimate value. If the parameters of a plurality of stress estimation models are stored in the stress estimation model storage unit 43, the stress estimation unit 18 may select a stress estimation model to be used, based on the attribute information regarding the subject and/or the type of the observation data S3 used for the stress estimation.

The stress estimation unit 18 may also perform display control of the display device 3 related to the calculated stress estimate value. For example, the stress estimation unit 18 generates a display signal S2 for displaying information on the calculated stress estimate value. Then, the stress estimation unit 18 supplies the display device 3 with the display signal S2, thereby causing the display device 3 to display the information on the stress estimate value. In another example, instead of performing the control to display the stress estimate value itself, or in addition to this, the stress estimation unit 18 may perform a control to display information on the stress level determined based on the comparison between the stress estimate value and a predetermined threshold value, and/or a control to display information on advice according to the stress level. The viewer of the display device 3 in this case, for example, may be the subject or may be a person who manages or supervises the stress state of the subject. Further, the stress estimation unit 18 may perform the audio output of the information on the stress estimate value by a sound output device (not shown).

Each component of the division unit 15, the short-term feature value calculation unit 16, the stress feature value calculation unit 17, and the stress estimation unit 18 described in FIG. 3 can be realized, for example, by the processor 11 executing a program. Additionally, the necessary programs may be recorded on any non-volatile storage medium and installed as necessary to realize each component. It should be noted that at least a portion of each of these components may be implemented by any combination of hardware, firmware, and software, without being limited to being implemented by software based on a program. At least some of these components may also be implemented using user programmable integrated circuit such as, for example, a FPGA (Field-Programmable Gate Array) and a microcontroller. In this case, the integrated circuit may be used to realize a program functioning as each of the above components. Further, at least a part of the components may be configured by ASSP (Application Specific Standard Produce), ASIC (Application Specific Integrated Circuit) or a quantum processor (quantum computer control chip). Thus, each of the above-described components may be realized by various hardware. Furthermore, each of these components may be implemented by cooperation of a plurality of computers, for example, using cloud computing technology.

### (4) Details of Short-Term Feature Value Calculation Unit

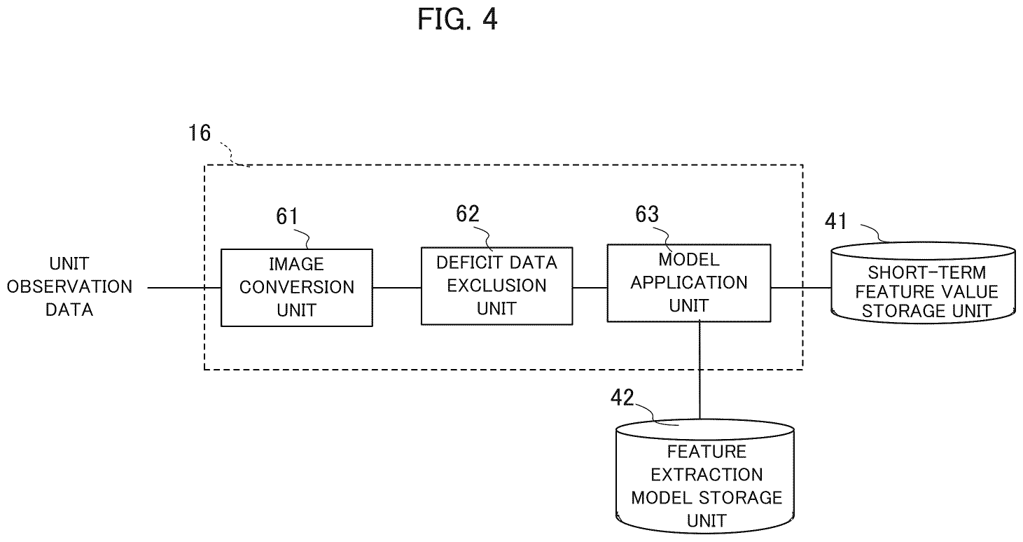

FIG. 4 is an example of a detailed functional block diagram of the short-term feature value calculation unit 16. The short-term feature value calculation unit 16 functionally includes an image conversion unit 61, a deficit data exclusion unit 62, and a model application unit 63.

The image conversion unit 61 converts each piece of unit observation data, which is generated by dividing the observation data S3, into a predetermined-size image (also referred to as “model input image”) which conforms to the input format of the feature extraction model. In this case, the image conversion unit 61 generates a spectrogram image representing the frequency characteristics of the unit observation data as a model input image. For example, the horizontal axis and the vertical axis of the spectrogram image are time and frequency, respectively, and the pixel value thereof is the amplitude (intensity) of frequency regarding the corresponding time and frequency. In this case, the image conversion unit 61 sets a window with reference to the time corresponding to the target column of determination of the pixel value and then applies the Fourier transform to the unit observation data in the window. Then, the image conversion unit 61 moves the above-described window according to the target column of determination of the pixel value, and calculates the pixel value (intensity of frequency) corresponding to each time (column). The above-described window is set to a length shorter than the time length of the unit observation data, and is set to a half of the time length of the unit observation data as an example. The model input image is an example of the “model input data”.

Thus, the image conversion unit 61 can convert each piece of unit observation data into data that conforms to the input format of the feature extraction model. Instead of generating a spectrogram image, the image conversion unit 61 may generate, from the unit observation data, an image representing a graph having the horizontal axis which represents the time and the vertical axis which represents the size of the observed value (index value) for each time, as the model input image. In some embodiments, the image conversion unit 61 may generate data in a tensor format with any dimension number, other than an image that is a two-dimensional tensor, by converting the unit observation data.

The deficit data exclusion unit 62 performs processing for detecting deficit data, which is data that does not properly represent the state of the subject, from the model input images generated by the image conversion unit 61 and excluding the identified deficit data. The deficit data is one or more model input images generated from the unit observation data (e.g., data in which observed values are constant) in which the subject has not been substantially observed. Then, if the stress estimation is carried out including the short-term feature values based on such deficit data, it causes the stress estimation accuracy to deteriorate. Taking the above into consideration, the deficit data exclusion unit 62 determines that any model input image satisfying a predetermined condition is deficit data and supplies model input images other than the deficit data to the model application unit 63. The above-described predetermined condition may be any condition that is regarded as deficit data. For example, it may be a condition (e.g., dispersion is less than a predetermined value or the like) based on the dispersion of the pixel values of the model input image. The model application unit 63 may detect the deficit data by using an identification model that is trained to identify whether or not an image inputted to the model is the deficit data. In this case, the learned parameters of the identification model described above are stored in the storage device 4 or the like, and the model application unit 63 inputs the spectrogram image to the identification model configured on the basis of the learned parameters, and determines whether or not the inputted spectrogram image the deficit data on the basis of the identification result outputted from the result identification model. The process of excluding the deficit data may of course be performed on the unit observation data.

The model application unit 63 inputs each of the model input images other than the deficit data supplied from the deficit data exclusion unit 62 to the feature extraction model configured by the learned parameters stored in the feature extraction model storage unit 42. Then, the model application unit 63 stores each feature value outputted by the feature extraction model in the short-term feature value storage unit 41 as the short-term feature values in association with the time information included in the corresponding unit observation data.

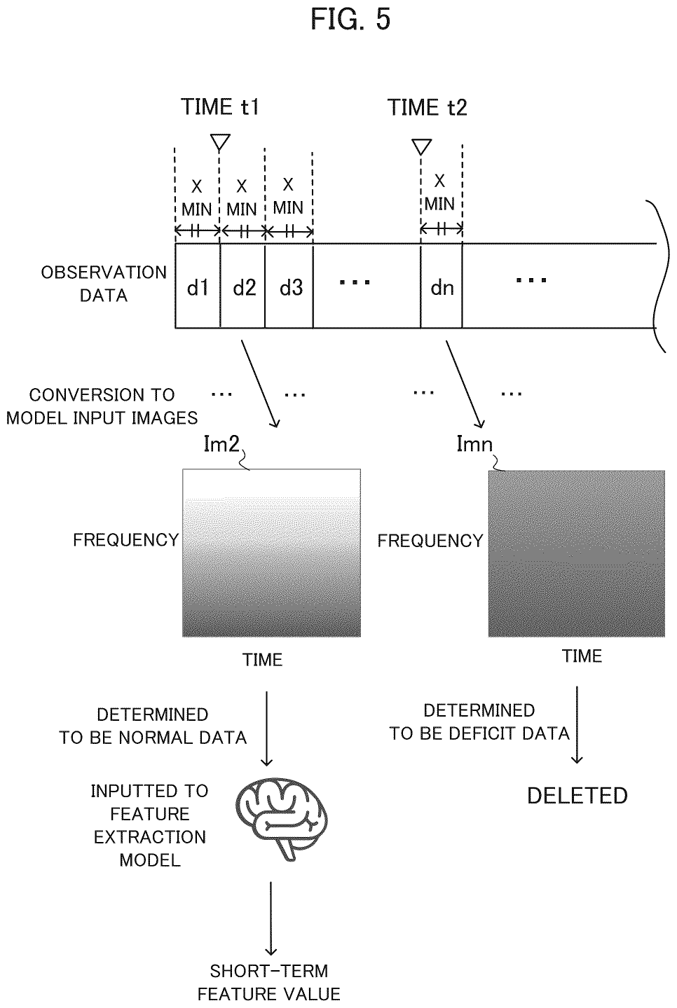

FIG. 5 is a diagram schematically showing a specific example of the process executed by the short-term feature value calculation unit 16. In this example, the observation data S3 having the horizontal axis as a time-axis is divided into multiple pieces of unit observation data including data “d1”, “d2”, “d3”, . . . , and “dn”, and the image conversion unit 61 converts each piece of the unit observation data into a model input image. Here, as a typical example of the model input image, there are provided a model input image “Im2” corresponding to the unit observation data d2, which is the observation data from the time “t1” with the length of x minutes (x is a positive number), and a model input image “Imn” corresponding to the unit observation data dn, which is the observation data from the time “t2” with the length of x minutes.

Then, the deficit data exclusion unit 62 determines that the model input image Im2 does not satisfy the above-described predetermined condition and is not deficit data (that is, normal data). Thus, the model application unit 63 calculates the short-term feature value by inputting the model input image Im2 into the already-learned feature extraction model. On the other hand, the deficit data exclusion unit 62 determines that the model input image Imn is the deficit data which satisfies the above-described predetermined condition, and deletes the model input image Imn without supplying it to the model application unit 63.

As described above, the short-term feature value calculation unit 16 converts the unit observation data into a model input image that matches the input format of the learned feature extraction model. Thereby, the short-term feature value calculation unit 16 can suitably generate the short-term feature values obtained by applying a feature extraction model used for image recognition or the like. Further, the short-term feature value calculation unit 16 can suppress generating the short-term feature values based on the unit observation data corresponding to deficit parts that do not properly represent the state of the subject in the observation data S3.

### (5) Processing Flow

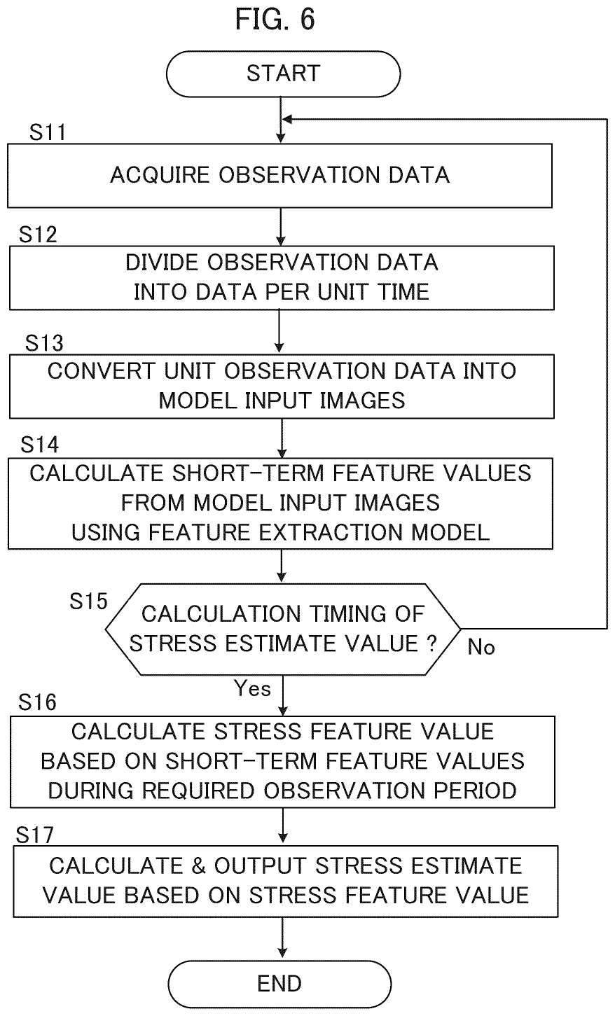

FIG. 6 is an example of a flowchart illustrating a procedure of a stress estimation process that is executed by the stress estimation device 1. The stress estimation device 1 repeatedly executes the process of the flowchart shown in FIG. 6.

First, the stress estimation device 1 acquires the observation data S3 from the sensor 5 (step S11). Then, the stress estimation device 1 divides the observation data S3 into plural pieces of unit observation data which is data per unit time (step S12). Then, the stress estimation device 1 converts each piece of unit observation data into a model input image (step S13). Then, the stress estimation device 1 calculates short-term feature values from respective model input images by using the feature extraction model configured by the learned parameters stored in the feature extraction model storage unit 42, and stores the calculated short-term feature values in the short-term feature value storage unit 41 in association with the time information (step S14). In this instance, the stress estimation device 1 may determine whether or not each model input image generated at step S13 is deficit data and exclude any model input images that are determined to be deficit data from the target of input to the feature extraction model.

Then, the stress estimation device 1 determines whether or not the calculation timing of the stress estimate value comes up (step S15). For example, upon detecting the user input for instructing the calculation of the stress estimate value, or upon determining that a predetermined calculation timing of the stress estimate value has come up, the stress estimation device 1 determines that the calculation timing of the stress estimate value comes up. Upon determining that the calculation timing of the stress estimate value does not come up (step S15; No), the stress estimation device 1 gets back to the process at step S11 and receives the observation data S3 and processes the received observation data S3.

Then, upon determining that the calculation timing of the stress estimate value comes up (step S15; Yes), the stress estimation device 1 calculates the stress feature value based on the short-term feature values corresponding to the observation data S3 generated during the required observation period (step S16). Then, the stress estimation device 1 calculates the stress estimate value based on the stress feature value calculated at step S16 and outputs information on the stress estimate value (step S17). In this case, the stress estimation device 1 inputs the stress feature value into the stress estimation model configured by the parameters stored in the stress estimation model storage unit 43. Then, the stress estimation device 1 outputs the stress estimate value of the subject which is a stress value outputted by the stress estimation model in response to the input.

### (6) Modifications

Hereinafter, preferred modifications of the above-described example embodiment will be described. The following modifications may be applied to the example embodiment described above in any combination.

### (First Modification)

The stress estimation device 1 may estimate the stress by using not only the short-term feature values based on the learned feature extraction model but also predetermined types of features extracted from the unit observation data as the short-term feature value.

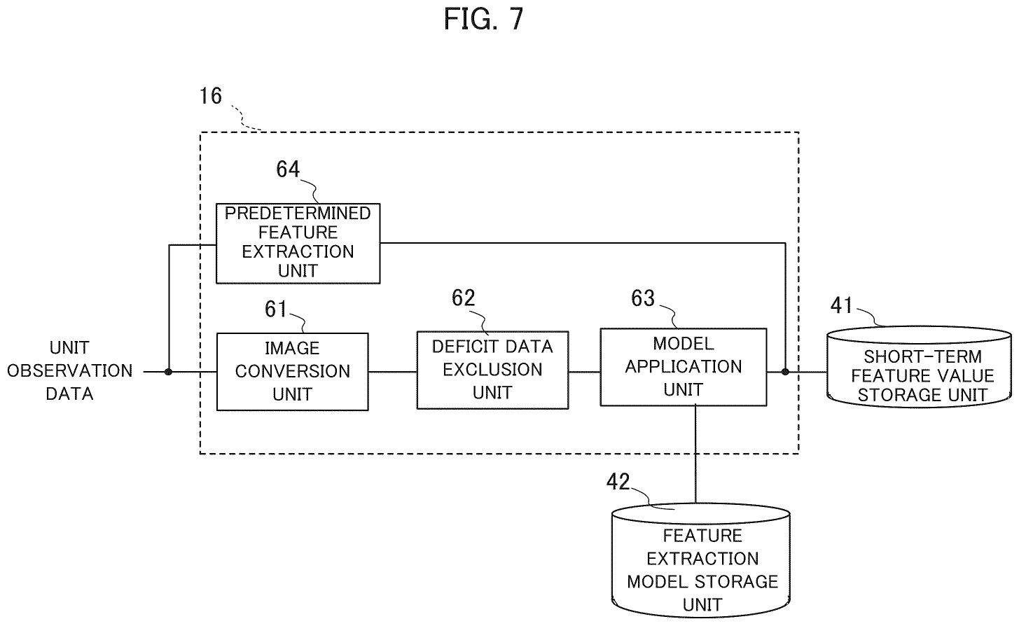

FIG. 7 is a functional block diagram of the short-term feature value calculation unit 16 according to the first modification. As shown in FIG. 7, the short-term feature value calculation unit 16 according to the modification includes an image conversion unit 61, a deficit data exclusion unit 62, a model application unit 63, and a predetermined feature extraction unit 64. The image conversion unit 61, the deficit data exclusion unit 62, and the model application unit 63 generate the short-term feature values by the same approach as in the above-described example embodiment.

The predetermined feature extraction unit 64 calculates predetermined types of features from each piece of unit observation data, and stores the calculated feature value as short-term feature value in the short-term feature value storage unit 41. Here, the predetermined feature extraction unit 64 calculates, as the feature value, for example, the average value of the unit observation data, the variance/standard deviation of the unit observation data, the maximum value of the unit observation data, the minimum value of the unit observation data, the quartile of the unit observation data, or any combination thereof. As such, the feature value calculated by the predetermined feature extraction unit 64 becomes predetermined types (that is, types determined in advance) of features extracted regardless of the learned feature extraction model. The predetermined feature value extraction unit 64 regards feature values which satisfy the predetermined condition among the calculated short-term feature values as feature values generated based on the unit observation data corresponding to deficit portions, and may discard the feature values without storing them in the short-term feature value storage unit 41.

Then, the short-term feature values calculated by the model application unit 63 and the short-term feature values calculated by the predetermined feature extraction unit 64 are stored in the short-term feature value storage unit 41 in association with same time information if the same unit observation data is used.

Here, the stress estimation method using these short-term feature values will be supplemented. In the first example, the stress feature value calculation unit 17 synthesizes the short-term feature values with which the same time information is associated by increasing the dimension number of the vector, and calculates the stress feature value using the synthesized short-term feature value. In this case, the dimension number of the synthesized short-term feature value is the sum of the dimension number of the short-term feature value calculated by the model application unit 63 and the dimension number of the short-term feature value calculated by the predetermined feature extraction unit 64. Thereafter, the stress estimation unit 18 calculates the stress estimate value by using the calculated stress feature value as input data of the stress estimation model.

In the second example, the stress feature value calculation unit 17 calculates the stress feature values from the short-term feature values calculated by the model application unit 63 and the stress feature values from the short-term feature values calculated by the predetermined feature extraction unit 64 and synthesizes these calculated stress feature values. Then, the stress estimation unit 18 calculates the stress estimate by using the synthesized stress feature values as input data of the stress estimation model. In this case, the dimension number of the synthesized stress feature values is the sum of the dimension number of the stress feature values based on the short-term feature values calculated by the model application unit 63 and the dimension number of the stress feature values based on the short-term feature values calculated by the predetermined feature extraction unit 64. In some embodiments, the stress estimation unit 18 calculates the stress values by applying the stress estimation model to the stress feature value based on the short-term feature values calculated by the model application unit 63 and the stress feature value based on the short-term feature values calculated by the predetermined feature extraction unit 64, respectively. Then, the stress estimation unit 18 calculates the average of the calculated stress values as the stress estimate value.

### (Second Modification)

In some embodiments, the stress estimation device 1 further performs processing for reducing the dimension number of feature values outputted by the feature extraction model and then calculates the feature values after the dimension reduction as the short-term feature values.

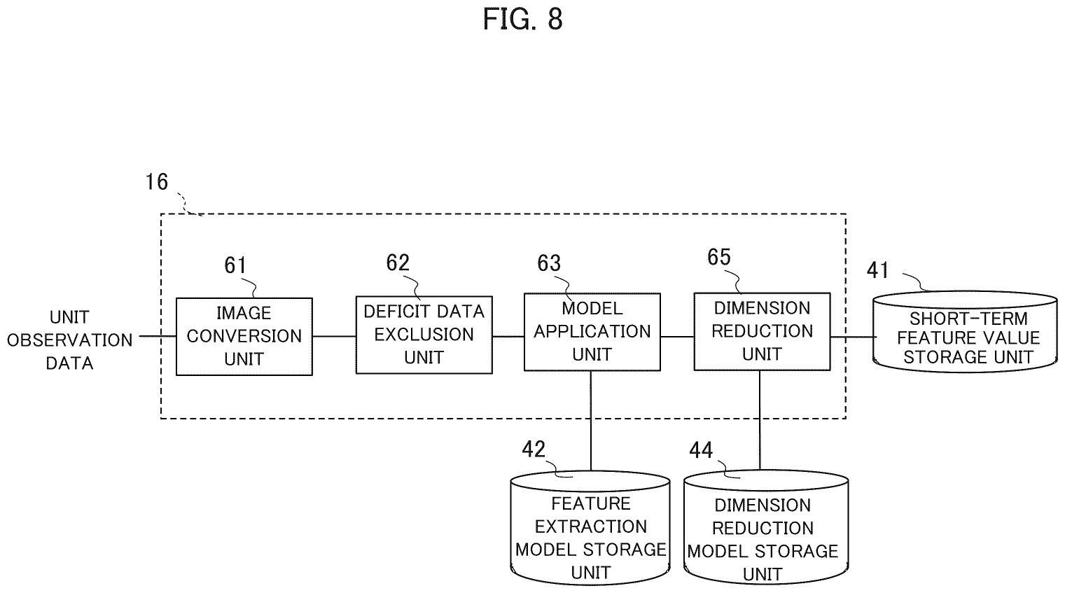

FIG. 8 is a functional block diagram of a short-term feature calculation unit 16 in the second modification. The short-term feature value calculation unit 16 according to the second modification includes an image conversion unit 61, a deficit data exclusion unit 62, a model application unit 63, and a dimension reduction unit 65. The image conversion unit 61, the deficit data exclusion unit 62, and the model application unit 63 perform the same processes as in the above-described example embodiment. The storage device 4 includes a dimension reduction model storage unit 44.

The dimension reduction unit 65 reduces the dimension number of the feature value calculated by the feature extraction model supplied from the model application unit 63. In this case, the dimension reduction model storage unit 44 stores the parameters of the dimension reduction model in advance. Then, the dimension reduction unit 65 inputs the feature value calculated by the feature extraction model to the dimension reduction model configured by the parameters stored in the dimension reduction model storage unit 44, and stores the dimension-reduced feature value outputted by the dimension reduction model in the short-term feature value storage unit 41 as the short-term feature value. In this case, the dimension reduction model in whose parameters are stored in the dimension reduction model storage unit 44 may be an arbitrary dimension reduction model. For example, the dimension reduction model storage unit 44 stores a transformation matrix, wherein the number of columns and the number of rows correspond to the dimension number of the feature value before the dimension reduction and the dimension number of the feature value after the dimension reduction, respectively.

According to this configuration, the short-term feature value calculation unit 16 can generate the short-term feature value with the reduced number of dimensions, thereby reducing the processing load at the stress feature value calculation unit 17 and the stress estimation unit 18.

The dimension reduction model whose parameters are stored in the dimension reduction model storage unit 44 may be trained in advance so as to perform dimension reduction suitable for stress estimation.

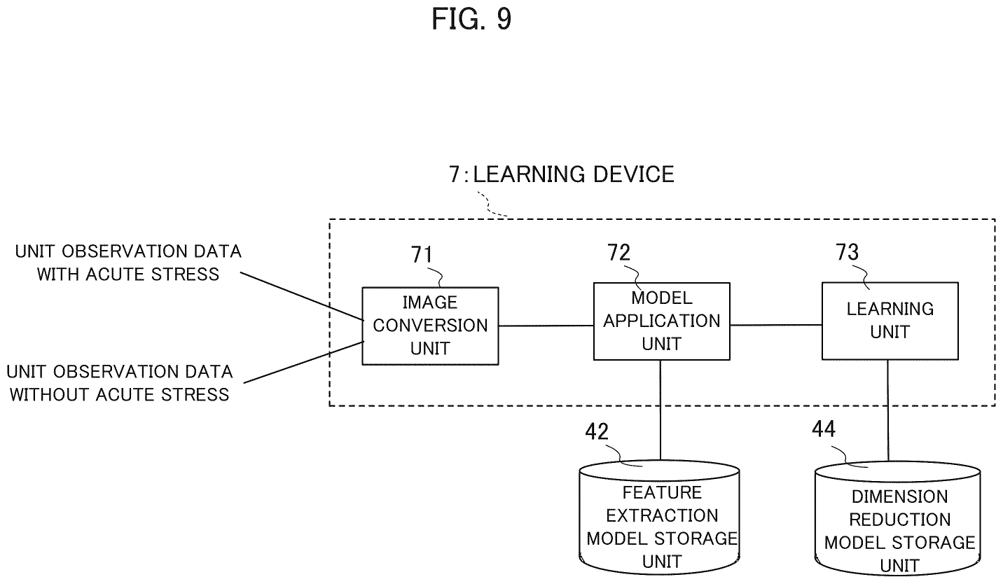

FIG. 9 is a functional block diagram related to the learning of a dimension reduction model. Here, the learning device 7 equipped with an image conversion unit 71, a model application unit 72, and a learning unit 73 performs learning of the dimension reduction model, and stores parameters of the dimension reduction model obtained by the learning in the dimension reduction model storage unit 44. The learning device 7 may be a stress estimation device 1 or may be a device other than the stress estimation device 1. In the former case, the processor 11 functions as the image conversion unit 71, the model application unit 72, and the learning unit 73. The image conversion unit 71 performs the same process as the image conversion unit 61 does, and the model application unit 72 performs the same process as the model application unit 63 does.

As training data, there are prepared unit observation data (also referred to as “under-stress data”) obtained by observing person(s) (who may not be the subject) in a condition in which acute stress is given to the person(s) and unit observation data (also referred to as “under-nonstress data”) obtained by observing person(s) in a condition in which acute stress is not given to the person(s). The number of samples of the under-stress data is set to be approximately the same number as the number of samples of the under-nonstress data, for example. The acute stress is stress in a relatively short term (a few minutes to a day or so). For example, the acute stress can be given to a person through a task such as solving a computational problem, speech in front of people, and pedaling with a cycling machine.

Then, the image conversion unit 71 converts each piece of the under-stress data and the under-nonstress data into an image, and the model application unit 72 inputs the above-described image to the feature extraction model configured by referring to the feature extraction model storage unit 42 to calculate the short-term feature values corresponding to the under-stress data and the under-nonstress data, respectively.

The learning unit 73 learns the parameters of the dimension reduction model based on the short-term feature values calculated by the model application unit 72. For example, the learning unit 73 applies a principal component analysis to all the short-term feature values corresponding to the under-stress data and the under-nonstress data which are training data, and obtains a transformation matrix for transforming the short-term feature values into data with reduced dimensions selected according to the magnitude of the variance. Thereafter, the parameters stored in the dimension reduction model storage unit 44 are referred to by the dimension reduction unit 65 shown in FIG. 8, and are used for dimension reduction of the short-term feature values in the stress estimation phase.

Thus, according to this modification, by performing the learning of the dimension reduction model using the under-stress data and the under-nonstress data, it is possible to train the dimension reduction model useful for the estimation of the chronic stress closely related to the acute stress.

### Second Example Embodiment

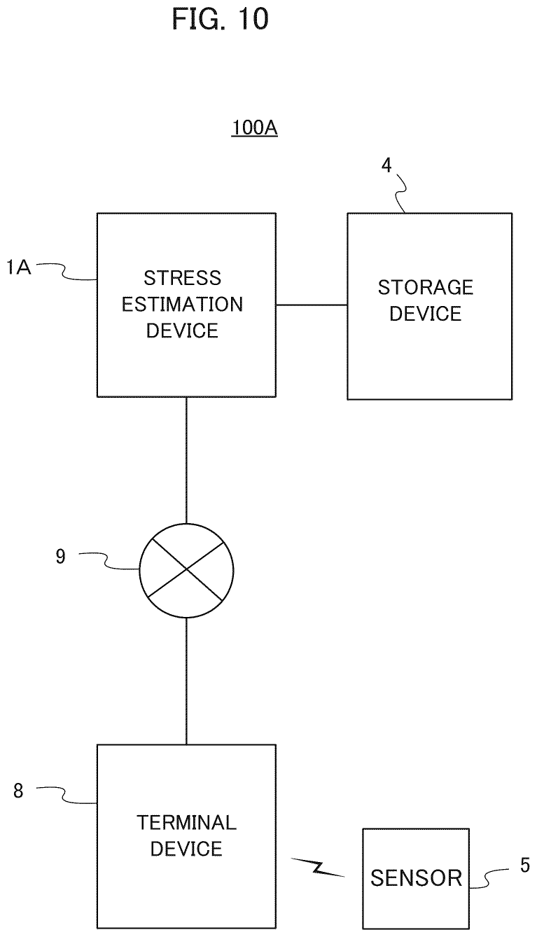

FIG. 10 shows a schematic configuration of a stress estimation 100A according to a second example embodiment. The stress estimation system 100A according to the second example embodiment includes a stress estimation device 1A that performs the same process as the stress estimation device 1 according to the first example embodiment, a storage device 4, and a terminal device 8 and a sensor 5 used by a subject. Hereinafter, the same components as in the first example embodiment are appropriately denoted by the same reference numerals, and a description thereof will be omitted.

In the second example embodiment, the stress estimation device 1A functions as a server and the terminal device 8 functions as a client. The stress estimation device 1A and the terminal device 8 perform data communication with each other via the network 9.

The terminal device 8 is a terminal used by the user serving as a subject, and is equipped with an input function, a display function, and a communication function, and functions as the input device 2 and the display device 3 shown in FIG. 1. The terminal device 8 may be, for example, a personal computer, a tablet-type terminal such as a smartphone, a PDA (Personal Digital Assistant), or the like. The terminal device 8 is electrically connected to the sensor 5 such as a wearable sensor worn by the user, and transmits the biological signal or the like (i.e., information corresponding to the observation data S3 in FIG. 1) of the subject outputted by the sensor 5 to the stress estimation device 1A through the network 9. Further, the terminal device 8 transmits information generated by the user input (information corresponding to the input signal S1 in FIG. 1) to the stress estimation device 1A. The sensor 5 may be incorporated in the terminal device 8. In some embodiments, the sensor 5 is equipped with the functions of the terminal device 8 and performs data communication with the stress estimation device 1A.

The stress estimation device 1A is equipped with the same hardware configuration as the hardware configuration of the stress estimation device 1 shown in FIG. 2, and the processor 11 of the stress estimation device 1A is equipped with the functional blocks shown in FIG. 3. The stress estimation device 1A receives information corresponding to the input signal S1 and the observation data S3 in FIG. 1 from the terminal device 8 via the network 9 and refers to various information stored in the storage device 4 to perform a stress estimation process for the subject. The stress estimation device 1A transmits an output signal for outputting the stress estimation result to the terminal device 8 through the network 9 in response to the display request from the terminal device 8.

Thus, the stress estimation device 1A according to the second example embodiment can estimate the stress condition of the subject based on the biological signal or the like of the subject received from the terminal used by the subject and suitably present the estimation result to the subject on the terminal.

### Third Example Embodiment

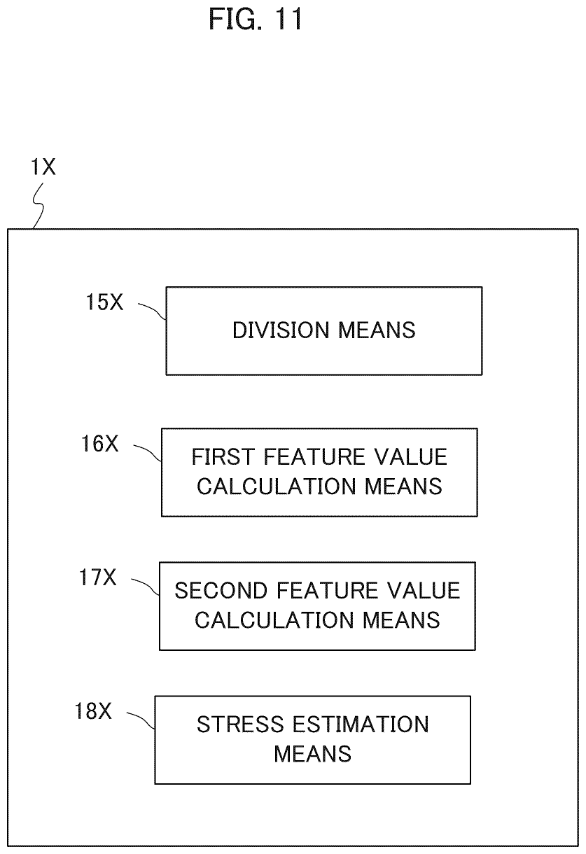

FIG. 11 is a block diagram of a stress estimation device 1X according to a third example embodiment. The stress estimating device 1X mainly includes a division means 15X, a first feature value calculation means 16X, a second feature value calculation means 17X, and a stress estimation means 18X. The stress estimation device 1X may be configured by a plurality of devices.

The division means 15X is configured to divide observation data representing chronological states of a subject. Examples of the division means 15X include the division unit in the first example embodiment (including modifications, hereinafter the same) and the division unit 15 in the second example embodiment. 15

The first feature value calculation means 16X is configured to calculate first feature values, which are feature values of divided observation data, based on a learned feature extraction model. The term “divided observation data” is, for example, unit observation data in the first example embodiment or the second example embodiment, and the term “first feature values” is, for example, short-term feature values in the first example embodiment or the second example embodiment. Examples of the first feature value calculation means 16X include the short-term feature value calculation unit 16 in the first example embodiment or the second example embodiment.

The second feature value calculation means 17X is configured to calculate a second feature value based on a plurality of the first feature values. Examples of the “second feature value” include the “stress feature value” in the first example embodiment or the second example embodiment. Examples of the second feature value calculation means 17X include the stress feature value calculation unit 17 in the first example embodiment or the second example embodiment.

The stress estimation means 18X is configured to estimate a stress of the subject based on the second feature value. Examples of the stress estimation means 18X include the stress estimation unit 18 in the first example embodiment or the second example embodiment.

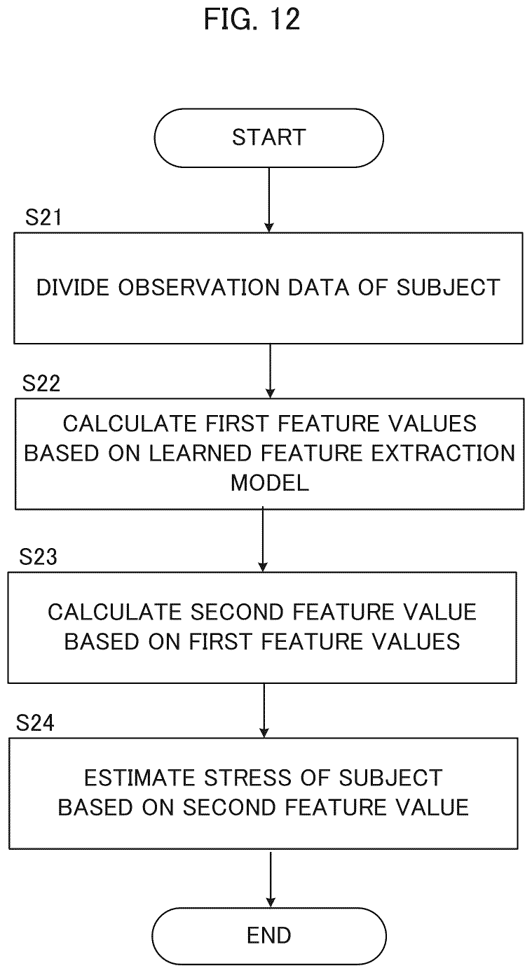

FIG. 12 is an example of a flowchart that is executed by the stress estimation device 1X in the third example embodiment. First, the division means 15X divides observation data representing chronological states of a subject (step S21). The first feature value calculation means 16X calculates first feature values, which are feature values of divided observation data, based on a learned feature extraction model (step S22). The second feature value calculation means 17X calculates a second feature value based on a plurality of the first feature values (step S23). The stress estimation means 18X estimates a stress of the subject based on the second feature value (step S24).

According to the third example embodiment, the stress estimation device 1X can calculate the first feature values useful for stress estimation using the learned feature extraction model and thereby estimate the stress of the subject with high accuracy.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above can be described as, but not limited to, the following Supplementary Notes.

### [Supplementary Note 1]

A stress estimation device comprising:
a division means configured to divide observation data representing chronological states of a subject;
a first feature value calculation means configured to calculate first feature values, which are feature values of the divided observation data, based on a learned feature extraction model;
a second feature value calculation means configured to calculate a second feature value based on a plurality of the first feature values; and
a stress estimation means configured to estimate a stress of the subject based on the second feature value.

### [Supplementary Note 2]

The stress estimation device according to Supplementary Note 1,
wherein the first feature value calculation means comprises:
   a conversion means configured to convert each piece of the divided observation data into model input data, which conforms to an input format of the feature extraction model,
   a model application means configured to input the model input data into the feature extraction model, and acquire a feature value outputted by the feature extraction model as the first feature value.

### [Supplementary Note 3]

The stress estimation device according to Supplementary Note 2,
wherein the conversion means is configured to generate a spectrogram image for each piece of the divided observation data as the model input data.

### [Supplementary Note 4]

The stress estimation device according to Supplementary Note 2 or 3,
wherein the first feature value calculation means further comprises
   a deficit data exclusion means configured to determine whether or not each piece of the model input data is deficit data with deficiency, and exclude the deficit data from a target of input to the feature extraction model.

### [Supplementary Note 5]

The stress estimation device according to any one of Supplementary Notes 1 to 4,
wherein the second feature value calculation means is configured to calculate the second feature value based on the first feature values corresponding to the observation data generated in a period which affects the stress.

### [Supplementary Note 6]

The stress estimation device according to any one of Supplementary Notes 1 to 5,
wherein the second feature value calculation means is configured to calculate the second feature value representing a statistic of the first feature values.

### [Supplementary Note 7]

The stress estimation device according to any one of Supplementary Notes 1 to 6,
wherein the first feature value calculation means is configured to reduce dimensions of a feature value outputted by the feature extraction model, and calculate the first feature value to be the feature value after dimension reduction.

### [Supplementary Note 8]

The stress estimation device according to Supplementary Note 7,
wherein the first feature value calculation means is configured to reduce dimensions of the feature value based on a dimension reduction model configured to reduce dimension of data inputted to the dimension reduction model, and
wherein the dimension reduction model is trained based on observation data generated in a stress state and observation data generated in a nonstress state.

### [Supplementary Note 9]

The stress estimation device according to any one of Supplementary Notes 1 to 8,
wherein the division means is configured to divide the observation data into unit observation data which is observation data per unit time.

### [Supplementary Note 10]

A stress estimation method executed by a computer, the stress estimation method comprising:
dividing observation data representing chronological states of a subject;
calculating first feature values, which are feature values of the divided observation data, based on a learned feature extraction model;
calculating a second feature value based on a plurality of the first feature values; and
estimating a stress of the subject based on the second feature value.

### [Supplementary Note 11]

A storage medium storing a program executed by a computer, the program causing the computer to:
divide observation data representing chronological states of a subject;
calculate first feature values, which are feature values of the divided observation data, based on a learned feature extraction model;
calculate a second feature value based on a plurality of the first feature values; and
estimate a stress of the subject based on the second feature value.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Stress estimation device
2 Input device
3 Display device
4 Storage device
5 Sensor
8 Terminal device
100, 100A Stress estimation system

What is claimed is:

1. A stress estimation device comprising:
at least one memory configured to store instructions; and
at least one processor configured to execute the instructions to:
divide observation data representing chronological states of a subject;
convert each piece of the divided observation data into a spectrogram image, which conforms to an input format of a feature extraction model learned by machine learning:
input the spectrogram image into the feature extraction model, and acquire feature values each outputted by the feature extraction model as first feature values,
which are feature values of the divided observation data;
calculate a second feature value based on a plurality of the first feature values; and
estimate a stress of the subject based on the second feature value.

2.-3. (canceled)

4. The stress estimation device according to claim 1,
wherein the at least one processor is configured to further execute the instructions to determine whether or not each spectrogram image is deficit data with deficiency, and exclude the deficit data from a target of input to the feature extraction model.

5. The stress estimation device according to claim 1,
wherein the at least one processor is configured to execute the instructions to calculate the second feature value based on the first feature values corresponding to the observation data generated in a period which affects the stress.

6. The stress estimation device according to claim 1,
wherein the at least one processor is configured to execute the instructions to calculate the second feature value representing a statistic of the first feature values.

7. The stress estimation device according to claim 1,
wherein the at least one processor is configured to execute the instructions to reduce dimensions of a feature value outputted by the feature extraction model, and calculate the first feature value to be the feature value after dimension reduction.

8. The stress estimation device according to claim 7,
wherein the at least one processor is configured to execute the instructions to reduce dimensions of the feature value based on a dimension reduction model configured to reduce dimension of data inputted to the dimension reduction model, and
wherein the dimension reduction model is trained based on observation data generated in a stress state and observation data generated in a nonstress state.

9. The stress estimation device according to claim 1,
wherein the at least one processor is configured to execute the instructions to divide the observation data into unit observation data which is observation data per unit time.

10. A stress estimation method executed by a computer, the stress estimation method comprising:
dividing observation data representing chronological states of a subject;
converting each piece of the divided observation data into a spectrogram image, which conforms to an input format of a feature extraction model learned by machine learning;
inputting the spectrogram image into the feature extraction model, and acquiring feature values each outputted by the feature extraction model as first feature values,
which are feature values of the divided observation data;
calculating a second feature value based on a plurality of the first feature values; and
estimating a stress of the subject based on the second feature value.

11. A non-transitory computer readable storage medium storing a program executed by a computer, the program causing the computer to:
divide observation data representing chronological states of a subject;
convert each piece of the divided observation data into a spectrogram image, which conforms to an input format of a feature extraction model learned by machine learning;
input the spectrogram image into the feature extraction model, and acquire feature values each outputted by the feature extraction model as first feature values,
which are feature values of the divided observation data;
calculate a second feature value based on a plurality of the first feature values; and
estimate a stress of the subject based on the second feature value.

12. The stress estimation device according to claim 1,
wherein the at least one processor is configured to execute the instructions to display information on a level of the estimated stress and information on advice to a user according to the level to assist the user to perform a decision making.
